(19) United States
(12) Patent Application Publication
(10) Pub. No.: US 2024/0138765 A1
(43) Pub. Date: May 2, 2024

(54) MEDICAL DEVICE WITH RETRACTABLE SENSOR

(71) Applicant: BOSTON SCIENTIFIC SCIMED, INC., Maple Grove, MN (US)

(72) Inventors: Andrew Brian Graveley, Shoreview, MN (US); Daniel J. Foster, Lino Lakes, MN (US); Katie Knowles, Providence, RI (US)

(73) Assignee: BOSTON SCIENTIFIC SCIMED, INC., Maple Grove, MN (US)

(21) Appl. No.: 18/496,706

(22) Filed: Oct. 27, 2023

Related U.S. Application Data
(60) Provisional application No. 63/420,500, filed on Oct. 28, 2022.

(51) Int. Cl. A61B 5/00; A61B 5/06; A61B 17/34

(52) U.S. Cl. CPC A61B 5/6848; A61B 5/062; A61B 17/3403; A61B 2562/164

(57) ABSTRACT

An example medical device includes a handle, an actuation mechanism coupled to the handle, and a delivery needle, wherein a proximal end of the delivery needle is coupled to a first end of the handle. A fluid delivery device is coupled to a second end of the housing, and a retractable sensor is positioned within the handle. The retractable sensor is configured to extend distally through a lumen of the delivery needle in a non-retracted position during placement of the delivery needle within a body of a patient, wherein actuation of the actuation mechanism causes the retractable sensor to retract proximally through the lumen of the delivery needle into the handle to a retracted position.

### CROSS-REFERENCE TO RELATED APPLICATIONS

This application claims the benefit of U.S. Provisional Patent Application Ser. No. 63/420,500 filed Oct. 28, 2022, the disclosure of which is incorporated herein by reference.

### TECHNICAL FIELD

The present disclosure pertains to medical devices, and methods for manufacturing medical devices. More particularly, the present disclosure pertains to a medical device having a retractable sensor within and/or attached thereto for medical device position tracking.

### BACKGROUND

Needles are commonly used to deliver therapies, aspirate fluid, or acquire tissue samples, particularly in the prostate. In most cases, the needles must be guided under ultrasound, where an operator may control a two-dimensional ultrasound probe with one hand and place the needle with the other.

Placing the needle under ultrasound is difficult and requires the operator to estimate spatial distances and orientation between the ultrasound probe inside a patient and the needle outside the patient. For example, in the case of a prostate biopsy, the operator first estimates the trajectory of the needle based on the ultrasound image without any direct indication of where the needle is located relative to anatomy shown on the ultrasound image. Once the needle is inserted through a perineum, the operator aligns an ultrasound imaging plane to the needle tip to visualize the tip and place the needle in a desired location. However, if the needle is oblique to the ultrasound imaging plane the operator may not see the needle tip, and many operators prefer an oblique approach so they can sample all regions of the prostate through a relatively smaller area of the perineum, and thus reduce the area they need to anesthetize prior to the procedure. Once the needle is visualized and placed in the desired location, the operator then estimates the three-dimensional trajectory of the needle when the needle advances forward from the retracted position to ensure a path that the needle will travel when advanced is only through tissue that can be safely biopsied. If the needle veers towards any critical anatomy, such as the rectal wall, urethra, seminal vesicles, or blood vessels, the needle may be advanced too quickly for the operator to track or correct course, causing the critical anatomy to be pierced, leading to complications for the patient. The use of a magnetic sensor may allow the operator to track the needle inside the body and avoid such complications. However, the use of a magnetic sensor may take up valuable space within a needle and impede the injection of viscous fluids or gels, and/or limit the access of other tools. Thus, an improved medical device may be desirable.

### BRIEF SUMMARY

This disclosure provides design, material, manufacturing method, and use alternatives for medical devices. An example medical device may include a handle, an actuation mechanism coupled to the handle, a delivery needle having a distal end, a proximal end, and a lumen extending from the distal end to the proximal end, wherein the proximal end of the delivery needle is coupled to a first end of the handle. A fluid delivery device may be coupled to a second end of the housing, and a retractable sensor may be positioned within the handle. The retractable sensor may be configured to extend distally through the lumen of the delivery needle in a non-retracted position during placement of the delivery needle within a body of a patient, wherein actuation of the actuation mechanism causes the retractable sensor to retract proximally through the lumen of the delivery needle into the handle to a retracted position.

Alternatively, or additionally to any of the embodiments above, when the retractable sensor is in the retracted position, the retractable sensor may be wound on a spool contained within the handle.

Alternatively, or additionally to any of the embodiments above, the fluid delivery device may be a syringe.

Alternatively, or additionally to any of the embodiments above, the fluid delivery device may be coupled to the second end of the handle via a luer-lock fitting.

Alternatively, or additionally to any of the embodiments above, when the retractable sensor is in the non-retracted position, a tab engages with the spool to lock the spool and thereby the retractable sensor in the non-retracted position.

Alternatively, or additionally to any of the embodiments above, actuation of the actuation mechanism may break the tab, thereby unlocking the spool, causing the retractable sensor to retract proximally and wind around the spool within the handle to the retraced position.

Alternatively, or additionally to any of the embodiments above, the retractable sensor may be housed within a flex-circuit cable.

Alternatively, or additionally to any of the embodiments above, the retractable sensor may have an outer diameter of Ø.018 inches.

Alternatively, or additionally to any of the embodiments above, upon retraction of the retractable sensor, the fluid delivery device is configured to deliver a fluid through the lumen of the delivery needle.

Alternatively, or additionally to any of the embodiments above, the retractable sensor is a position sensor.

Alternatively, or additionally to any of the embodiments above, the actuation mechanism is a button.

Another example medical device may include a handle, an actuation mechanism coupled to the handle, a delivery needle having a distal end, a proximal end, and a lumen extending from the distal end to the proximal end, wherein the proximal end of the delivery needle is coupled to a first end of the handle, and a retractable sensor positioned within the handle. The retractable sensor may be configured to extend distally through the lumen of the delivery needle in a non-retracted position during placement of the delivery needle within a body of a patient, and a spool may be positioned within the handle, the retractable sensor coupled to the spool. Actuation of the actuation mechanism may cause the retractable sensor to retract proximally through the lumen of the delivery needle into the handle and wind around the spool in a retracted position.

Alternatively, or additionally to any of the embodiments above, when the retractable sensor is in the non-retracted position, a tab may engage with the spool to lock the spool and thereby the retractable sensor in the non-retracted position.

Alternatively, or additionally to any of the embodiments above, actuation of the actuation mechanism may break the tab, thereby unlocking the spool, causing the retractable sensor to retract proximally and wind around the spool within the handle to the retracted position.

Alternatively, or additionally to any of the embodiments above, the actuation mechanism may be a button.

Alternatively, or additionally to any of the embodiments above, the retractable sensor may be a position sensor.

Another example medical device may include a handle, an actuation mechanism coupled to the handle, and a delivery needle having a distal end, a proximal end, and a lumen extending from the distal end to the proximal end, wherein the proximal end of the delivery needle is coupled to a first end of the handle. A retractable sensor may be positioned within the handle, the retractable sensor configured to extend distally through the lumen of the delivery needle in a non-retracted position during placement of the delivery needle within a body of a patient.

Alternatively, or additionally to any of the embodiments above, actuation of the actuation mechanism may cause the retractable sensor to retract proximally through the lumen of the delivery needle into the handle to a retracted position.

Alternatively, or additionally to any of the embodiments above, the actuation mechanism may be a button.

Alternatively, or additionally to any of the embodiments above, when the retractable sensor is in the retracted position, the retractable sensor may be wound on a spool contained within the handle.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1A illustrates an exemplary medical device including a delivery needle and a fluid delivery device;

FIG. 1B illustrates an exploded view of the exemplary medical device including the delivery needle and the fluid delivery device, as in FIG. 1A;

FIG. 2 illustrates an exploded view of an exemplary handle of the exemplary medical device as in FIG. 1A;

FIG. 3A illustrates a side view of the exemplary handle of the exemplary medical device, as in FIG. 1A, wherein a second portion of the handle is not shown.

FIG. 3B illustrates a side perspective view of the exemplary handle of the exemplary medical device, as in FIG. 3A, wherein a first portion of the handle and the second portion of the handle are not shown;

FIG. 4 illustrates a side view of the exemplary handle including the delivery needle of the exemplary medical device, as in FIG. 1A, wherein a second portion of the handle is transparent;

FIG. 5 illustrates a cross-section view of the exemplary handle including the delivery needle of, as in FIG. 4, taken at line 5-5, wherein an actuation mechanism is in a released position;

FIG. 6A illustrates a cross-section view of the exemplary handle including the delivery needle of, as in FIG. 4, taken at line 5-5, wherein the actuation mechanism is in a depressed position;

FIG. 6B illustrates an enlarged view of a portion of the actuation mechanism as in FIG. 6A, taken at circle 6B;

FIG. 7 illustrates an exemplary medical device including a delivery needle and a fluid delivery device;

FIG. 8 illustrates a perspective view of the exemplary medical device including the delivery needle, as in FIG. 7, wherein the fluid delivery device is not shown;

FIG. 9 illustrates a side view of the exemplary medical device including the delivery needle and the fluid delivery device, as in FIG. 7, wherein an electrical cable is not shown;

FIG. 10A illustrates a cross-section view the exemplary medical device including the delivery needle and the fluid delivery device, as in FIG. 9, taken at line 10A-10A;

FIG. 10B illustrates an enlarged view of a portion of a first valve, as in FIG. 10A, taken at circle 10B;

FIG. 10C illustrates an enlarged view of a distal end of the delivery needle as in FIG. 10A, taken at circle 10C;

FIG. 11 illustrates a side view of the exemplary medical device including the delivery needle, as in FIG. 7, wherein an adapter is disengaged from a handle;

FIG. 12A illustrates a cross-section view of the exemplary medical device including the delivery needle wherein the adapter is disengaged from the handle, as in FIG. 11, taken at line 12A-12A;

FIG. 12B illustrates an enlarged view of a portion of a second valve, as in FIG. 12A, taken at circle 12B;

FIG. 13 illustrates a rear perspective view of the exemplary medical device including the delivery needle, as in FIG. 7, wherein an adapter is disengaged from a handle;

FIG. 14 illustrates a front perspective view of the exemplary medical device including the delivery needle, as in FIG. 14, wherein the adapter is fully removed from the handle; and

FIG. 15 illustrates a partial cross-section view of the exemplary medical device including the delivery needle, as in FIG. 11, wherein a housing of the handle is shown in cross-section.

While the disclosure is amenable to various modifications and alternative forms, specifics thereof have been shown by way of example in the drawings and will be described in detail. It should be understood, however, that the intention is not to limit the disclosure to the embodiments described. On the contrary, the intention is to cover all modifications, equivalents, and alternatives falling within the spirit of the disclosure.

### DETAILED DESCRIPTION

For the following defined terms, these definitions shall be applied, unless a different definition is given in the claims or elsewhere in this specification.

All numeric values are herein assumed to be modified by the term “about,” whether explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes, 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in this specification to “an embodiment”, “some embodiments”, “other embodiments”, etc., indicate that the embodiment described may include one or more features, structures, and/or characteristics. However, such recitations do not necessarily mean that all embodiments include the features, structures, and/or characteristics. Additionally, when features, structures, and/or characteristics are described in connection with one embodiment, such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Needles are commonly used to deliver therapy, aspirate fluid, or sample tissue. In most cases, needles must be guided under ultrasound, wherein a user may control a two-dimensional ultrasound probe with one hand and place the needle with the other. Placing the needle under ultrasound may be difficult and ay require the user to estimate spatial distances and orientation between the tissue displayed in the ultrasound image and the needle as it penetrates the tissue. This coordination may be especially difficult when the ultrasound transducer is far from the access point, such as during prostate procedures.

Guiding the needle under two-dimensional ultrasound may require the user to continuously move and rotate the ultrasound probe to find the position of the needle tip and understand its trajectory. If the user fails to find and identify the needle under ultrasound, or misinterprets a partial view of the needle, the user may unintentionally pierce critical structures. Magnetic tracking offers the ability to track tool tips anywhere inside the body using a sensor. While magnetic sensors are small and may fit within the needle, the sensor may impede injection of viscous fluids or gels, or other access tools. Thus, an improved medical device for delivering fluids and/or gels may be desirable.

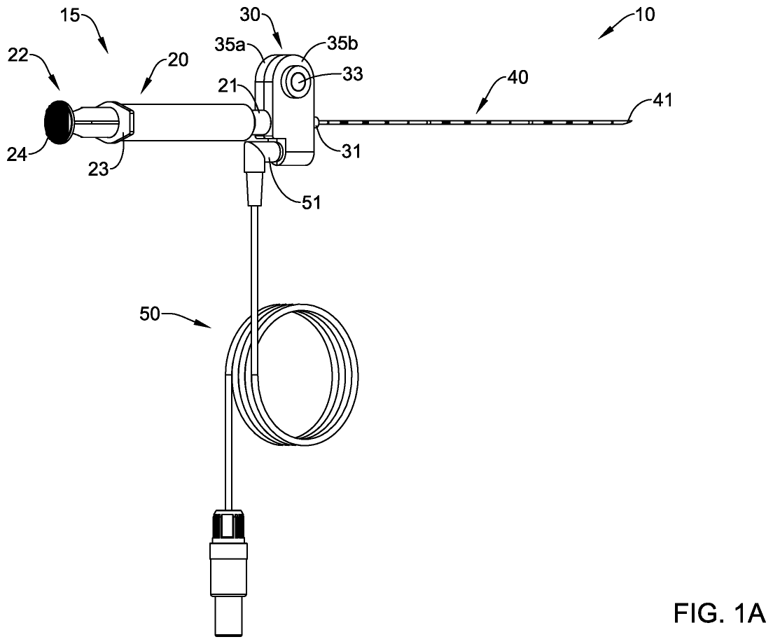

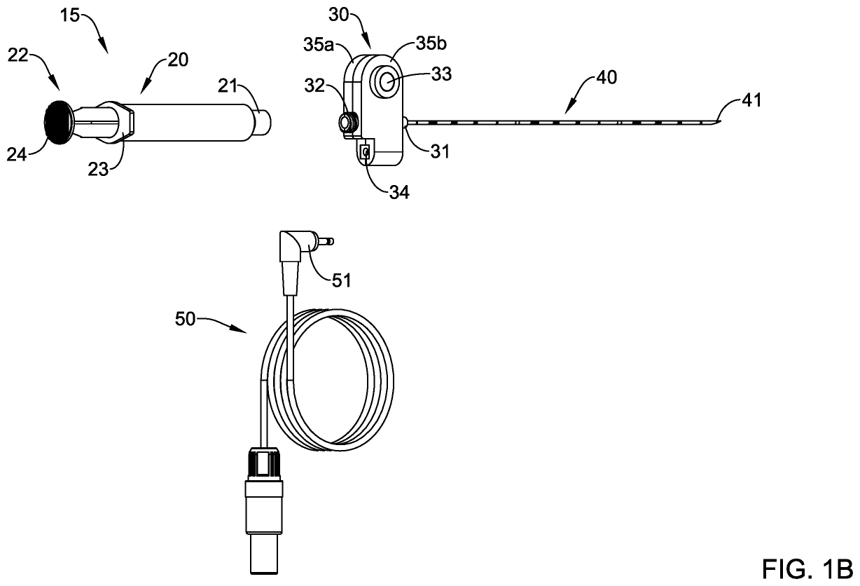

FIG. 1A illustrates an exemplary medical device 10 including a delivery needle 40 and a fluid delivery device 15. FIG. 1B illustrates an exploded view of the exemplary medical device 10 including the delivery needle 40 and the fluid delivery device 15. As shown in FIGS. 1A to 1B, the medical device 10 may be a delivery device including a handle 30, the delivery needle 40, the fluid delivery device 15, and a cable 50. The handle 30 may include a first portion 35a and a second portion 35b, and an actuation mechanism 33 coupled to the handle 30. In some cases, the actuation mechanism 33 may include a button. In some cases, the actuation mechanism 33 may include a lever, a turn crank, a trigger, or any other suitable actuation mechanism. The handle 30 may further include a first end 31 and a second end 32. The delivery needle 40 may include a distal end 41 and a proximal end (not explicitly shown in FIG. 1A or 1B), and the proximal end of the delivery needle 40 may be coupled to the first end 31 of the handle 30. The delivery needle 40 may be coupled to the handle 30 via adhesive bonding, laser welding, resistance welding, insert injection molding, or any other suitable method of attachment. In some cases, the delivery needle 40 may be beveled at the distal end 41 to enhance tissue penetration.

In some cases, the fluid delivery device 15 may be a syringe 20. The syringe 20 may include a first end 21 and a second end 22. The first end 21 of the syringe 20 may be removably coupled to the second end 32 of the handle 30 via a luer lock. In some cases, the first end 21 of the syringe 20 may be removably coupled to the second end 32 of the handle 30 via a snap fit, an interference fit, a luer slip, or any other suitable method of attachment. In some cases, the syringe 20 may be configured to contain saline to, for example, flush a lumen of the handle 30 and the delivery needle 40, and/or the targeted tissue, prior to delivering a therapy. In some cases, the syringe 20 may be configured to contain saline for hydro-dissection, for example. In such cases, the saline contained within the syringe 20 may be used to prime the delivery needle 40 to remove air from a lumen of the delivery needle 40 to prevent air from entering tissue and obscuring an ultrasound image. Once the delivery needle 40 is in a desired location, the saline is injected to perform hydro-dissection of the tissue. In some cases, the syringe 20 may configured to contain a fluid mixture, such as, for example, water and polyethylene glycol (PEG). In some cases, the syringe 20 may be configured to contain any other type of fluid as desired. The second end 22 of the syringe 20 may include a grip 23 and a plunger 24. In use, a user may hold the grip 23 and translate the plunger 24 in a distal direction to administer a fluid (e.g., saline, or a fluid mixture) contained within the syringe 20.

The cable 50 may be removably coupled to the handle 30, via an electrical port 34. In such cases, the cable 50 may include a barrel connector 51 which may be configured to be plugged into the electrical port 34. In some cases, the cable 50 may be configured to be coupled to a controller (not shown) which may receive signals, for example, from a sensor located within the delivery needle 40, and/or transmit signals, for example, to a transmitter device.

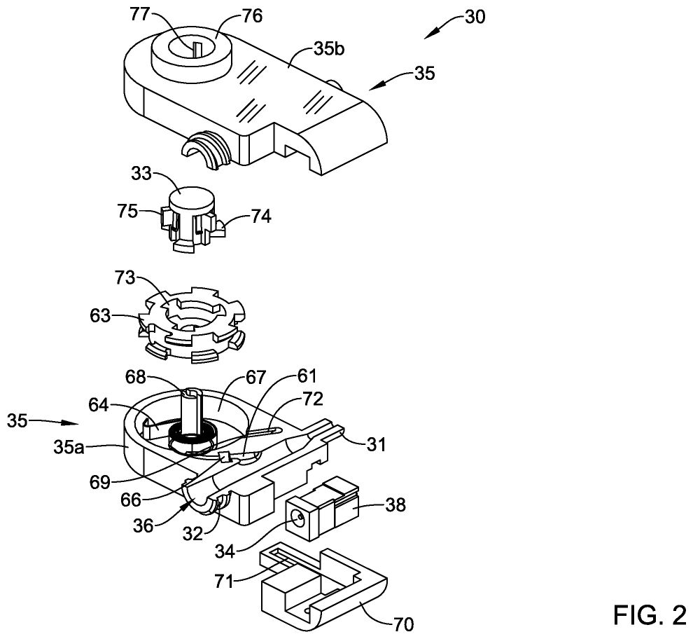

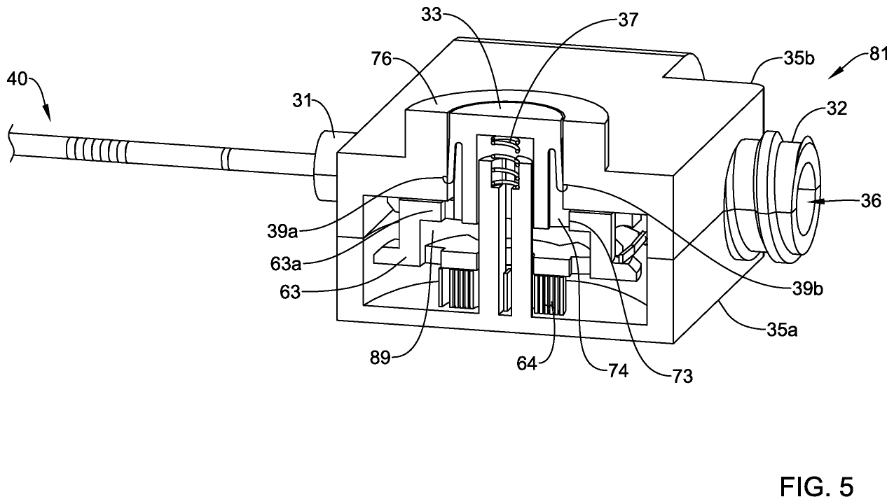

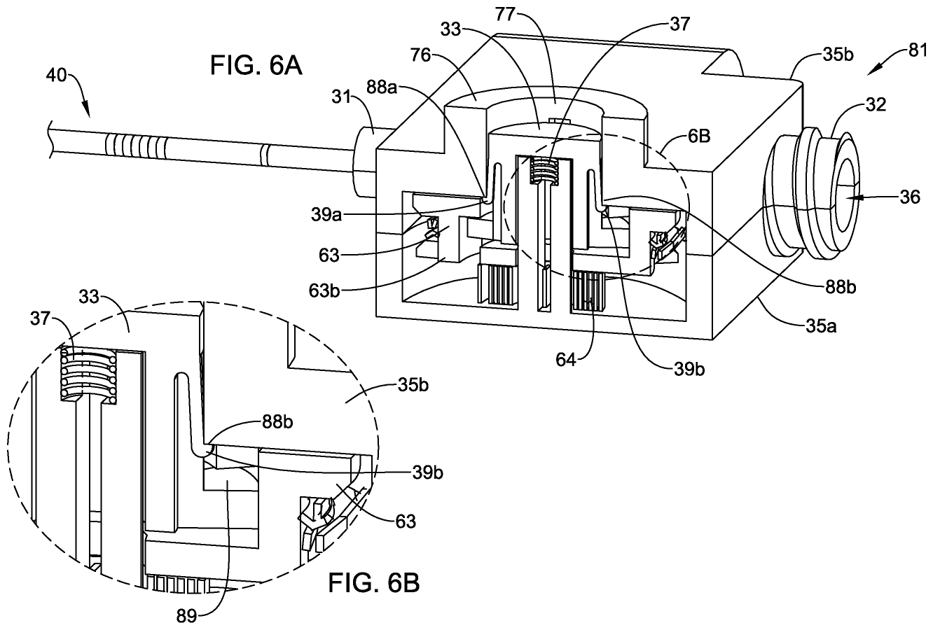

FIG. 2 illustrates an exploded view of the exemplary handle 30 of the exemplary medical device 10 as in FIGS. 1A to 1B. The handle 30 may include the first end 31 and the second end 32 and a lumen 36 extending from the first end 31 to the second end 32. The handle may further include the first portion 35a and the second portion 35b. While the second portion 35b is shown as transparent in FIG. 2, it may be contemplated that the second portion 35b may be formed from an opaque material. The first portion 35a of the handle 30 may include a cavity 67 that extends within the first portion 35a of the handle 30. While not explicitly shown in FIG. 2, it may be appreciated that the second portion 35b may include a second cavity that is positioned opposite the cavity 67 when the handle 30 is fully assembled. A shaft 68 may be positioned within the cavity 67 of the handle 30, and a torsion spring 64 may be positioned around the shaft 68 within the cavity 67. A spool 63 may further be configured to be positioned around the shaft 68 and reside within the cavity 67, such that the torsion spring 64 may be positioned between the spool 63 and the first portion 35a of the handle 30. The spool 63 may include one or more notches 73 which may be configured to engage with one or more tabs 74 of the actuation mechanism 33, which will be discussed further with reference to FIGS. 5 to 6B.

The actuation mechanism 33 may include the one or more tabs 74 and one or more stops 75. The actuation mechanism 33 may be configured to fit over the shaft 68, and the one or more tabs 74 of the actuation mechanism 33 may be configured to align with the one or more notches 73 when the actuation mechanism 33 is placed over the shaft 68. The actuation mechanism 33 may be configured to extend through an opening 77 within the second portion 35b of the handle 30. An outer rim 76 on the second portion 35b of the handle 30 may be positioned around the opening 77, and thereby the actuation mechanism 33 when the handle 30 is fully assembled.

The first portion 35a of the handle 30 may include a channel 72 configured to permit a proximal end of a sensor cable (not explicitly shown in FIG. 2) to extend therethrough. The channel 72 may extend from the cavity 67 within the first portion 35a of the handle 30 through to a cable lumen 71 within an electrical housing 70. The electrical housing 70 may be configured to house an electrical component 38, which may include the electrical port 34. The electrical component 38 may receive signals, for example, from a sensor located within the delivery needle 40, and/or transmit signals, for example, to a transmitter device. The electrical housing 70 may be configured to attach to the first portion 35a of the handle 30 via a snap fit, interference fit, adhesive bonding, laser welding, resistance welding, insert injection molding, or any other suitable method of attachment.

The first portion 35a of the handle 30 may further include a sensor channel 69 configured to extend from the cavity 67 within the first portion 35a of the handle 30 through to the lumen 36 of the handle 30. A valve 66 may be positioned within the sensor channel 69 between the cavity 67 and the lumen 36 of the handle 30. The valve 66 may be configured to provide a seal between the lumen 36 and the sensor channel 69 to prevent a fluid within the lumen 36 from entering the sensor channel 69 and/or to prevent air from the sensor channel 69 from entering the lumen 36, and thereby the delivery needle 40.

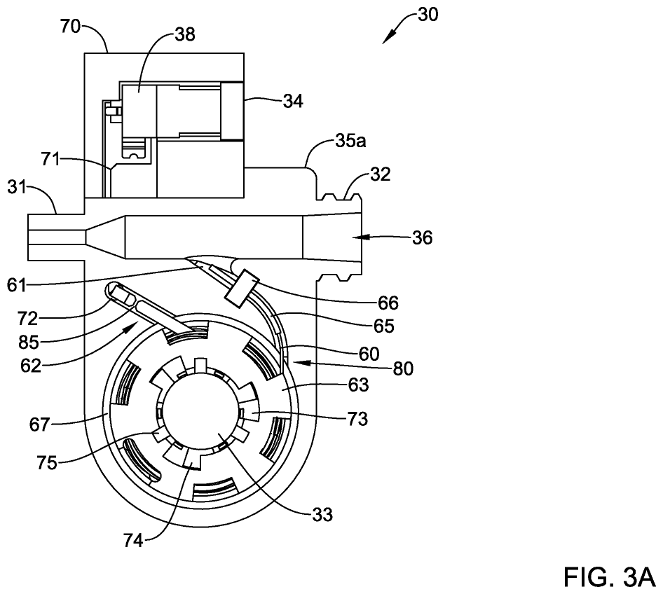

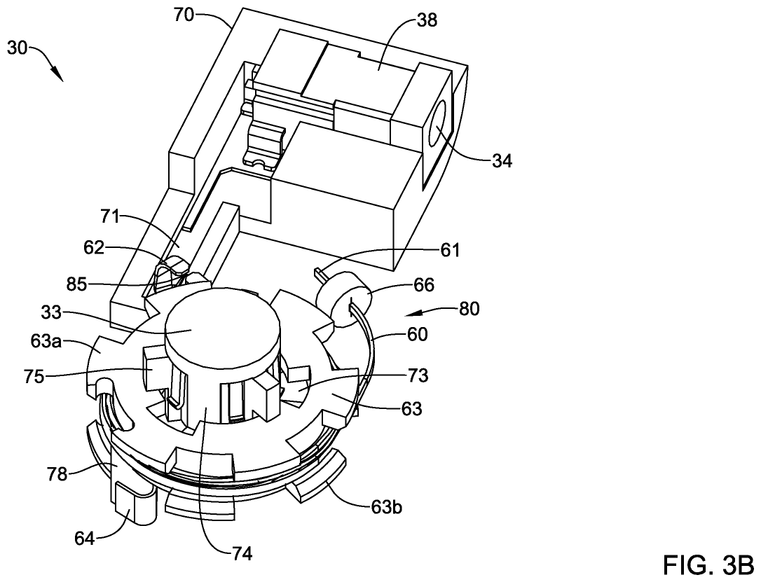

FIG. 3A illustrates a side view of the exemplary handle of the exemplary medical device, as in FIG. 1A, wherein the second portion 35b of the handle 30 is not shown, and FIG. 3B illustrates a side perspective view of the exemplary handle 30 of the exemplary medical device 10, as in FIG. 3A, wherein the first portion 35a of the handle 30 and the second portion 35b of the handle 30 are not shown. As shown in FIG. 3A, the handle 30 may include the first end 31 and the second end 32 and the lumen 36 extending from the first end 31 to the second end 32. The handle 30 may further include the first portion 35a. The first portion 35a of the handle 30 may include the cavity 67 that extends within the first portion 35a of the handle 30. The spool 63 may be configured to be positioned within the cavity 67. The spool 63 may include the one or more notches 73 which may be configured to engage with the one or more tabs 74 of the actuation mechanism 33.

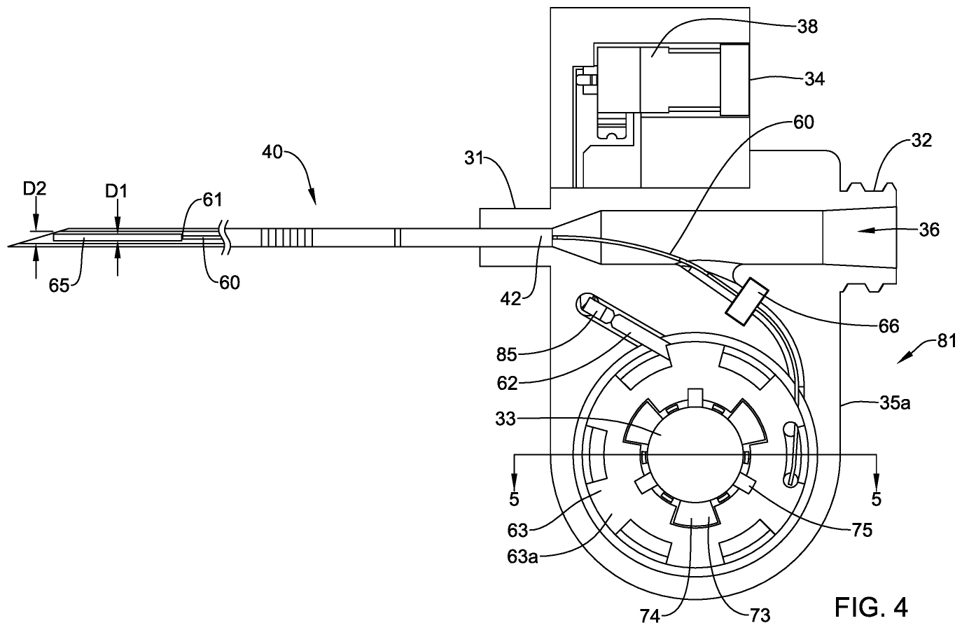

As shown in FIGS. 3A to 3B, a retractable sensor cable 60 may be coupled to the spool 63 positioned within the handle 30. In some cases, the retractable sensor cable 60 may be a flex-circuit cable. For example, in some cases, the retractable sensor cable 60 may include a two-layer flex circuit, having one conductive trace per layer and a stiffening member. In some cases, the retractable sensor cable 60 may include a twisted pair cable or a micro coax cable. The retractable sensor cable 60 may include a distal end 61 and a proximal end 62. The proximal end 62 of the retractable sensor cable 60 may be positioned within the channel 72 and may extend through to the cable lumen 71 within the electrical housing 70. The proximal end 62 of the retractable sensor cable 60 may be operatively connected via a thin, flex circuit, to the electrical component 38 within the electrical housing 70. The proximal end 62 of the retractable sensor cable 60 may include a portion positioned between the spool 63 and the electrical component 38 which may include a perforated portion 85. Upon actuation of the actuation mechanism 33, the one or more tabs 74 disengage from the one or more notches 73, thereby allowing the spool 63 to rotate. The rotation of the spool 63 breaks the perforated portion 85 (as shown in FIGS. 3A to 3B) of the proximal end 62, thereby unlocking the spool 63, and allowing the spool 63 to continue to rotate and the retractable sensor cable 60 to wind onto the spool 63 to a retracted position 80. The distal end 61 of the retractable sensor cable 60 may be configured to extend distally through a lumen of the delivery needle (e.g., delivery needle 40) in a non-retracted position (shown in FIG. 4) during placement of the delivery needle within a body of a patient. The distal end 61 of the retractable sensor cable 60 may include a sensor 65. In some cases, the sensor 65 may be position sensor, such as an electromagnetic sensor or an optical sensor. In some cases, the sensor 65 may be an anisotropic-magneto-resistive sensing element, a giant-magneto-resistive element, a tunneling-magneto-resistive sensing element, a colossal-magneto-resistive sensing element, an extraordinary-magneto-resistive sensing element, an inductive sensor, a planar coil sensor, a fluxgate, a Hall-effect sensing element, a spin hall sensing element, a giant-magneto impedance sensor, a magnetic gradiometer, or the like.

As shown in FIGS. 3A to 3B, the retractable sensor cable 60 may be in the retracted position 80 wherein the retractable sensor cable 60 is wound around the spool 63. The retractable sensor cable 60 may be configured to pass through the valve 66. The valve 66 may be configured to provide a seal between the lumen 36 and the sensor channel 69 to prevent a fluid within the lumen 36 from entering the sensor channel 69 and/or to prevent air from the sensor channel 69 from entering the lumen 36 of the handle 30.

As shown in FIG. 3B, the actuation mechanism 33 may include the one or more tabs 74 and the one or more stops 75. The one or more tabs 74 of the actuation mechanism 33 may be configured to align with the one or more notches 73 of the spool 63. The torsion spring 64 may be configured to engage with a post 78 positioned at a second surface 63b of the spool 63. When the actuation mechanism 33 is actuated, for example, depressed, the one or more tabs 74 move past the one or more notches 73 of the spool 63 and into an open space within the spool 63. The one or more stops 75 may be configured to engage with a first surface 63a of the spool 63, thereby preventing the actuation mechanism 33 from advancing further within the spool 63. When the one or more tabs 74 move into the open space within the spool 63, the rotational energy stored in the torsion spring 64 is released, thereby causing the spool 63 to rotate. This will be discussed in further detail with reference to FIGS. 5 to 6B.

FIG. 4 illustrates a side view of the exemplary handle 30 including the delivery needle 40 of the exemplary medical device 10, as in FIG. 1A, wherein the second portion 35b of the handle 30 is transparent and the distal end 41 of the delivery needle 40 is shown in cross-section. As shown in FIG. 4, the handle 30 may include the first end 31 and the second end 32 and the lumen 36 extending from the first end 31 to the second end 32. The handle 30 may further include the first portion 35a. The first portion 35a of the handle 30 may include the cavity 67 that extends within the first portion 35a of the handle 30. The spool 63 may be configured to be positioned within the cavity 67. The spool 63 may include the one or more notches 73 which may be configured to engage with the one or more tabs 74 of the actuation mechanism 33.

The retractable sensor cable 60 may be coupled to the spool 63 positioned within the handle 30. The retractable sensor cable 60 may include the distal end 61 and the proximal end 62. The proximal end 62 of the retractable sensor cable 60 may include a portion positioned between the spool 63 and the electrical component 38 which may include the perforated portion 85. In some cases, the portion positioned between the spool 63 and the electrical component 38 may not include the stiffening member, thereby permitting the perforated portion 85 to tear more easily. The distal end 61 of the retractable sensor cable 60 may include the sensor 65. In some cases, the sensor 65 may be inductive, using a coiled conductor wrapped around a high-permeability core, with end of the coil wire attached to the retractable sensor cable 60. The sensor 65 may enable a position and/or an orientation of the distal end 41 of the delivery needle 40 to be tracked. In some cases, the sensor 65 may facilitate tracking of a position and/or an orientation of the distal end 41 of the delivery needle 40 relative to an ultrasound imaging plane such that the position and/or the orientation of the delivery needle 40 may be displayed in an imaging plane, although this is not shown. In some cases, the sensor 65 may include an outer diameter D₁ within a range of Ø.005 inches to Ø.149 inches. In some cases, the sensor 65 may include an outer diameter D₁ of Ø.018 inches, or any other suitable outer diameter. In some cases, the delivery needle 40 may include an outer diameter D₂ within a range of Ø.038 inches to Ø.150 inches. In some cases, the delivery needle 40 may include an outer diameter D₂ of Ø.050 inches, or any other suitable outer diameter. The outer diameter of the sensor 65 is minimal relative to the outer diameter of the delivery needle 40 to allow for a fluid to flow through a lumen 45 of the delivery needle 40 without requiring excessing force and pressure applied to the syringe 20.

The delivery needle 40 may include the distal end 41, a proximal end 42, and the lumen 45 extending from the distal end 41 to the proximal end 42. The proximal end 42 of the delivery needle 40 may be coupled to the first end 31 of the handle 30. The delivery needle 40 may be coupled to the handle 30 via adhesive bonding, laser welding, resistance welding, insert injection molding, or any other suitable method of attachment. In some cases, the delivery needle 40 may be beveled at the distal end 41 to enhance tissue penetration.

As shown in NG. 4, the retractable sensor cable 60 may be in a non-retracted position 81. When the retractable sensor cable 60 is in the non-retracted position 81, the distal end 41 of the retractable sensor cable 60 may be configured to extend distally through the lumen 45 of the delivery needle 40, such that the sensor 65 positioned at the distal end 61 of the retractable sensor cable 60 may be positioned within the lumen 45 near the distal end 41 of the delivery needle 40 during placement of the delivery needle 40 within a body of a patient. When the retractable sensor cable 60 is in the non-retracted position 81, the actuation mechanism 33 may be in a released position. The one or more tabs 74 of the actuation mechanism 33 may be configured to align with the one or more notches 73 of the spool 63. The one or more tabs 74 may lie within the one or more notches 73 and may be flush with the first surface 63a of the spool 63. When the one or more tabs 74 are positioned within the one or more tabs 74, the spool 63 is locked in place such that the spool 63 cannot rotate. When the retractable sensor cable 60 is in the non-retracted position 81, the perforated portion 85 of the retractable sensor cable 60 may remain intact (e.g., the perforation is not broken).

FIG. 5 illustrates a cross-section view of the exemplary handle 30 including the delivery needle 40, as in FIG. 4, taken at line 5-5, wherein the actuation mechanism 33 is in a released position. FIG. 6A illustrates a cross-section view of the exemplary handle 30 including the delivery needle 40, as in FIG. 4, taken at line 5-5, wherein the actuation mechanism 33 is in a depressed position. As shown in FIG. 5, the actuation mechanism 33 is in the released position. When the actuation mechanism 33 is in the released position, the one or more tabs 74 of the actuation mechanism 33 may be configured to engage with the spool 63 to lock the spool 63 and thereby lock the retractable sensor cable 60 in the non-retracted position 81. For example, the one or more tabs 74 may be configured to align with the one or more notches 73 of the spool 63, wherein the one or more tabs 74 may lie within the one or more notches 73 and may be flush with the first surface 63a of the spool 63. When the one or more tabs 74 are positioned within the one or more tabs 74, the spool 63 is locked in place such that the spool 63 cannot rotate. Thus, holding the retractable sensor cable 60 in the non-retracted position 81. In some cases, a compression spring 37 may exert a force against the actuation mechanism 33, thereby holding the actuation mechanism 33 in the released position. In some cases, rather than the compression spring 37, a living hinge or a detent feature may be used to hold the actuation mechanism 33 in the released position. The retractable sensor cable 60 is held in the non-retracted position 81 during the placement of the delivery needle 40 within the body of the patient.

As shown in FIG. 6A, the actuation mechanism 33 is in the depressed position. The one or more tabs 74 of the actuation mechanism 33 may be configured to align with the one or more notches 73 of the spool 63. The torsion spring 64 may be configured to engage with the post 78 (not shown in FIG. 6A) positioned at the second surface 63b of the spool 63. When the actuation mechanism 33 is actuated, for example, depressed, the one or more tabs 74 move past the one or more notches 73 of the spool 63 and into an open space 89 within the spool 63. The one or more stops 75 may be configured to engage with a first surface 63a of the spool 63, thereby preventing the actuation mechanism 33 from advancing further within the spool 63. Further, a first latch 39a and a second latch 39b may engage with a first lip 88a and a second lip 88b, respectively. The first latch 39a and the second latch 39b may engage with the first lip 88a and the second lip 88b via a snap-fit. This snap-fit holds the actuation mechanism 33 in the depressed position. While it is shown that there are two latches, it may be contemplated that there may be one latch, three latches, five latches, seven latches, or any other suitable number of latches. When the one or more tabs 74 move into the open space within the spool 63, the rotational energy stored in the torsion spring 64 is released, thereby causing the spool 63 to rotate. In some cases, instead of rotational energy, rotation of the spool 63 may use electromagnetic energy, air and/or fluid pressure, piezoelectric energy, and or any other suitable type of energy.

In use, actuation of the actuation mechanism 33 may cause the retractable sensor cable 60 to retract proximally through the lumen 45 of the delivery needle 40, into the handle 30 and through the lumen 36 of the handle 30. The retractable sensor cable 60 may then automatically wind around the spool 63 in the retracted position 80. Upon retraction of the retractable sensor cable 60 and the sensor 65, the fluid delivery device 15 (e.g., the syringe 20) may be configured to deliver a fluid through the lumen 45 of the delivery needle 40.

FIG. 6B illustrates an enlarged view of a portion of the actuation mechanism 33 as in FIG. 6A, taken at circle 6B. As shown in FIG. 6B, the second latch 39b engages with the second lip 88b of the opening 77. The second latch 39b locks the actuation mechanism 33 in the depressed position thereby allowing the spool 63 to rotate.

While it is illustrated that the actuation mechanism 33 is a button that may be depressed or released, it may be contemplated that the actuation mechanism 33 may include a turn crank in which the spool 63 may be manually rotated, thereby winding the retractable sensor cable 60 around the spool 63.

In another embodiment, instead of a delivery needle (e.g., delivery needle 40), the handle 30 including the spool 63 may be used in a rigid or a flexible endoscope or a catheter.

In another embodiment, a rotary union or a slip ring electrical connector may be used to allow the sensor (e.g., sensor 65) to stay connected while the spool (e.g., spool 63) rotates, and/or a region of cable that is wound counter to the spool 63 and thus unwinds when the sensor is retracted to stay attached to the electrical component (e.g., electrical component 38). This may allow a user to reuse the sensor by pulling the sensor back out of the handle (e.g., handle 30). In some cases, when another type of energy source, the energy may be reversed to wind and unwind the retractable sensor cable (e.g., retractable sensor cable 60).

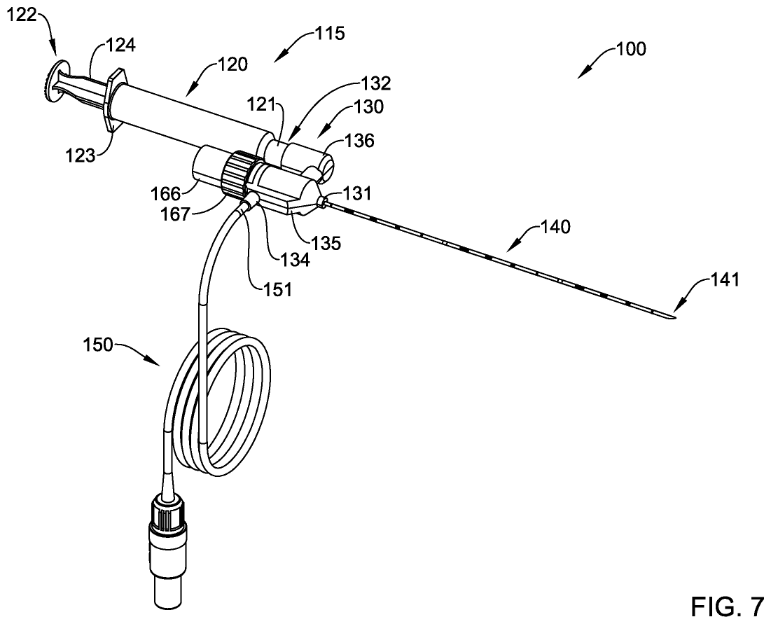

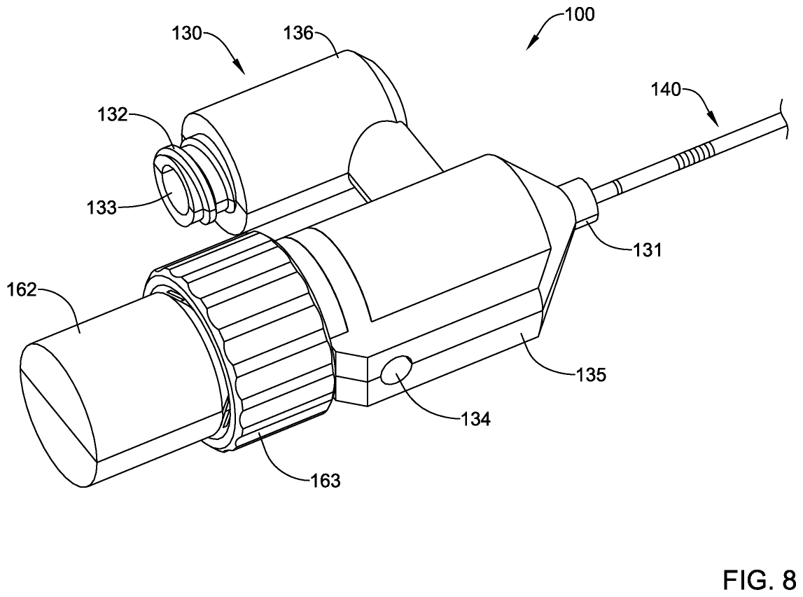

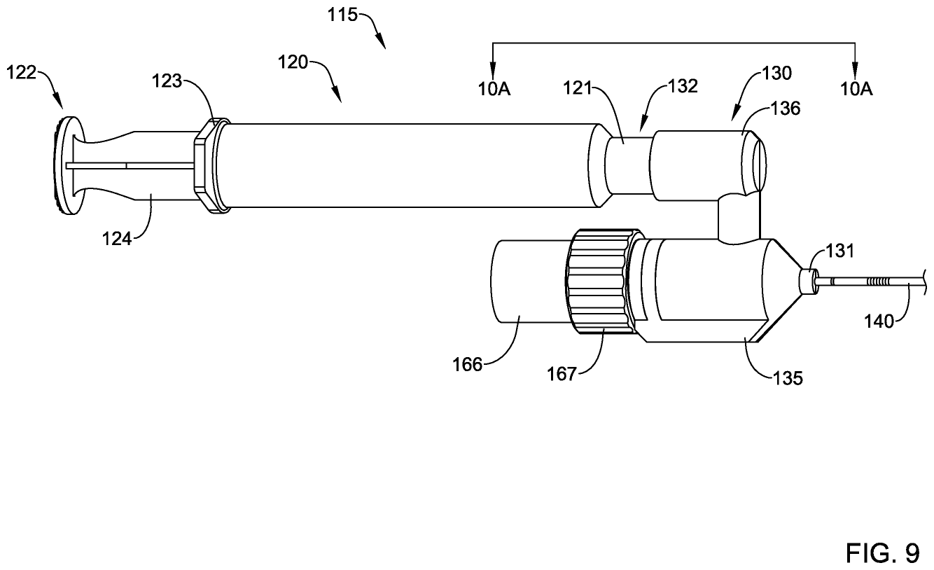

FIGS. 7 to 9 illustrate an exemplary medical device 100. FIG. 7 illustrates the exemplary medical device 100 including a delivery needle 140 and a fluid delivery device 115. FIG. 8 illustrates a side view of the exemplary medical device 100 including the delivery needle 140, as in FIG. 7, wherein the fluid delivery device 115 and the cable 150 are not shown, and FIG. 9 illustrates a side view of the exemplary medical device 100 including the delivery needle 140, as in FIG. 7, wherein the cable 150 is not shown. As shown in FIGS. 7 to 9, the medical device 100 may be a delivery device including a handle 130, the delivery needle 140, the fluid delivery device 115, and a cable 150. The handle 130 may include a first portion 135 and a second portion 136. The handle 130 may further include a first end 131 and a second end 132. As shown in FIG. 8, the second portion 136 of the handle 130 may include a first lumen 133. The delivery needle 140 may include a distal end 141 and a proximal end (not explicitly shown in FIG. 7), and the proximal end of the delivery needle 140 may be coupled to the first end 131 of the handle 130. The delivery needle 140 may be coupled to the handle 130 via adhesive bonding, laser welding, resistance welding, insert injection molding, or any other suitable method of attachment. In some cases, the delivery needle 140 may be beveled at the distal end 141 to enhance tissue penetration.

In some cases, the fluid delivery device 115 may be a syringe 120. The syringe 120 may include a first end 121 and a second end 122. The first end 121 of the syringe 120 may be removably coupled to the second end 132 of the handle 130 via a luer lock. In some cases, the first end 121 of the syringe 120 may be removably coupled to the second end 132 of the handle 130 via a snap fit, an interference fit, a luer slip, or any other suitable method of attachment. In some cases, the syringe 120 may be configured to contain saline to, for example, flush a lumen of the handle 130 and the delivery needle 140, and/or the targeted tissue, prior to delivering a therapy. In some cases, the syringe 120 may be configured to contain saline for hydro-dissection, for example. In such cases, the saline contained within the syringe 120 may be used to prime the delivery needle 140 to remove air from a lumen of the delivery needle 140 to prevent air from entering tissue and obscuring an ultrasound image. Once the delivery needle 140 is in a desired location, the saline is injected to perform hydro-dissection of the tissue. In some cases, the syringe 120 may configured to contain a fluid mixture, such as, for example, water and polyethylene glycol (PEG). In some cases, the syringe 120 may be configured to contain any other type of fluid as desired. The second end 122 of the syringe 120 may include a grip 123 and a plunger 124. In use, a user may hold the grip 123 and translate the plunger 124 in a distal direction to administer a fluid (e.g., saline, or a fluid mixture) contained within the syringe 120.

The cable 150 may be removably coupled to the handle 130, via an electrical port 134. In such cases, the cable 150 may include an electrical connector 151 which may be configured to be plugged into the electrical port 134. In some cases, the cable 150 may be configured to be coupled to a controller (not shown) which may receive signals, for example, from a sensor located within the delivery needle 40, and/or transmit signals, for example, to a transmitter device.

An adapter 166 may include an outer ring 167 and may be removably coupled to the first portion 135 of the handle 130 via a thread fastening mechanism. The outer ring 167 may include inner threads (not explicitly shown in FIG. 7) and may be configured to engage with outer threads on the first portion 135 of the handle 130. In some cases, the adapter 166 may be removably coupled to the first portion 135 of the handle 130 via snap-fit, interference fit, adhesive, or any other suitable method of attachment.

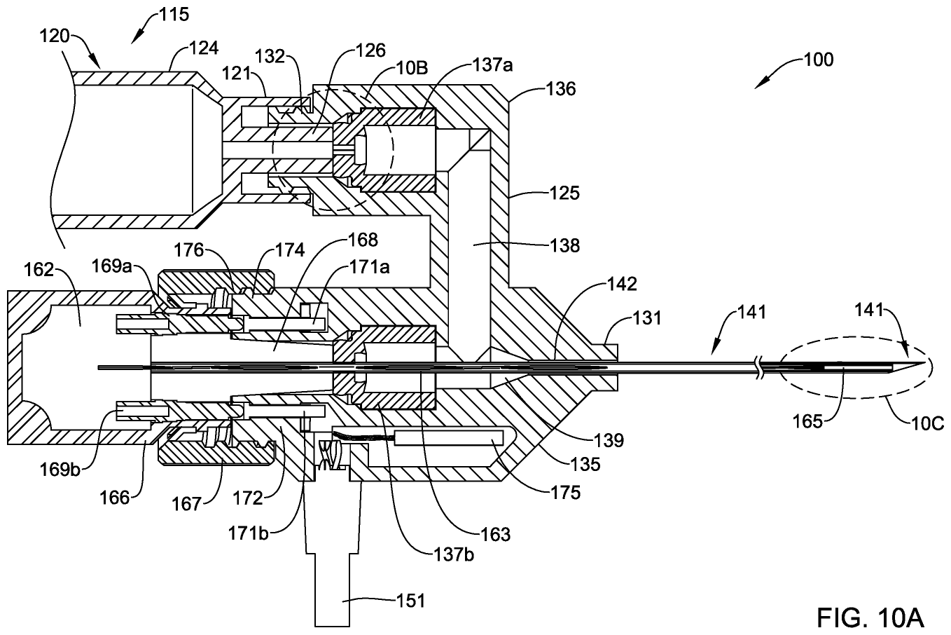

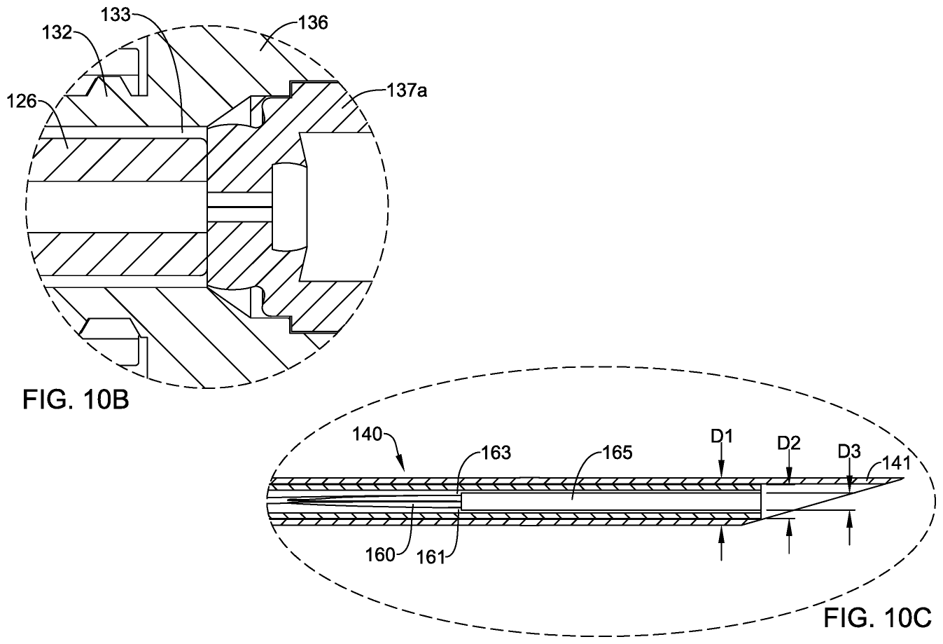

FIG. 10A illustrates a cross-section view the exemplary medical device 100 including the delivery needle 140 and the fluid delivery device 115, as in FIG. 9, taken at line 10A-10A. As discussed with reference to FIGS. 7 to 9, the handle 130 may include the first portion 135 and the second portion 136. The second portion 136 of the handle 130 may include the first lumen 133, and the first lumen 133 may connect with a second lumen 138 that passes from the second portion 136 of the handle 130 to the first portion 135 of the handle 130 through a bridge portion 125 of the handle 130. The second lumen 138 may connect with a third lumen 139 of the first portion 135 of the handle 130. The third lumen 139 may extend between the first end 131 of the handle 130 to an outlet 172 of the first portion 135 of the handle 130. The outlet 172 may include an opening configured to engage with the adapter 166. The outlet 172 may include outer threads 174 which may be configured to engage with inner threads 176 on the outer ring 167 of the adapter 166. The inner threads 176 and the outer threads 174 may engage via thread fastening to couple the adapter 166 to the first portion 135 of the handle 130. In some cases, the outlet 172 and the outer ring 167 may not include threads and may instead couple the adapter 166 and the first portion 135 of the handle 130 via interference fit, a snap-fit, adhesive, or any other suitable method of coupling. In some cases, the adapter 166 and the handle 130 may be formed as a single monolithic piece.

A sensor cable 160 may include a distal end 161 and a proximal end 162. The proximal end 162 of the sensor cable 160 may be coupled to the adapter 166 and may be configured to extend distally through the third lumen 139 and further through a lumen 145 of the delivery needle 140. In some cases, a stylet 163 may be configured to house the sensor cable 160. In some cases, the stylet 163, and thus the sensor cable 160 may be coupled to the adapter 166 such that when the adapter 166 is uncoupled from the outlet 172 of the first portion 135 of the handle 130, the stylet 163 including the sensor cable 160 is withdrawn (e.g., retracted) proximally from the lumen 145 of the delivery needle 140 and the third lumen 139 of the handle 130. In some cases, the sensor cable 160 may be a retractable sensor cable. A sensor 165 may be positioned at the distal end 161 of the sensor cable 160, such that when the stylet 163 including the sensor cable 160 extends through the lumen 145 of the delivery needle 140, the sensor 165 may be positioned within the lumen 145 near the distal end 141 of the delivery needle 140. In some cases, the sensor 165 may be held in position using a medical grade epoxy. In some cases, the sensor 165 may be held in position via a friction or interference fit. In some cases, the sensor 165 may be position sensor, such as an electromagnetic sensor or an optical sensor. In some cases, the sensor 65 may be an anisotropic-magneto-resistive sensing element, a giant-magneto-resistive element, a tunneling-magneto-resistive sensing element, a colossal-magneto-resistive sensing element, an extraordinary-magneto-resistive sensing element, an inductive sensor, a planar coil sensor, a fluxgate, a Hall-effect sensing element, a spin hall sensing element, a giant-magneto impedance sensor, a magnetic gradiometer, or the like. The sensor 165 may enable a position and/or an orientation of a distal end 141 of the delivery needle 140 to be tracked. In some cases, the sensor 165 may facilitate tracking of a position and/or an orientation of the distal end 141 of the delivery needle 140 relative to an ultrasound imaging plane such that the position and/or the orientation of the delivery needle 140 may be displayed in an imaging plane, although this is not shown.

The adapter 166 may include a tip 168 that may be configured to engage with the outlet 172 of the first portion 135 of the handle 130. The tip 168 and the outer ring 167 may engage with the outlet 172 in similar fashion to a luer lock. The tip 168 may be inserted into the third lumen 139 and may abut a second valve 137b that is positioned within the third lumen 139. The stylet 163 including the sensor cable 160 may be configured to pass through the second valve 137b that is positioned within the third lumen 139. The second valve 137b may be configured to provide a seal around the stylet 163 including the sensor cable 160, thereby preventing air from entering the third lumen 139 and/or a fluid from exiting the third lumen 139. In some cases, the second valve 137b may be a self-sealing valve, and may be configured to provide a seal between the outlet 172 and the third lumen 139 upon removal of the adapter 166, and thus the stylet 163 including the sensor cable 160 and the sensor 165. In some cases, the stylet 163 may be coupled to the tip 168 of the adapter 166. The stylet 163 may be coupled to the tip 168 via adhesive bonding, laser welding, resistance welding, insert injection molding, or any other suitable method of attachment.

In some cases, the adapter 166 may include a first pin 169a and a second pin 169b that may be configured to engage with a first socket 171a and a second socket 171b of the outlet 172 of the handle 130, respectively. Thus, when the tip 168 is inserted into the opening of the outlet 172, the first and second pins 169a, 169b may engage with and snap into the first and second sockets 171a, 171b, respectively. In some cases, the first pin 169a and the second pin 169b may form an electrical connection with the first socket 171a and the second socket 171b, respectively, like a plug into an outlet. In some cases, the proximal end 162 of the sensor cable 160 may be configured to pass through a portion of the stylet 163 that is coupled to the tip 168 of the adapter 166 and engage with the first and second pins 169a, 169b to couple the sensor cable 160 to the adapter 166.

In some cases, a second sensor 175 may be housed within the first portion 135 of the handle 130. In some cases, the second sensor 175 may be a position sensor, such as an electromagnetic sensor or an optical sensor. The second sensor 175 may monitor movement of the handle, and/or estimate a position and/or an orientation of a distal end 141 of the delivery needle 140 to be tracked, once the stylet 163 including the sensor cable 160 and the sensor 165 have been removed from the lumen 145 of the delivery needle 140. In some cases, the second sensor 175 may not be included.

As discussed, the syringe 120 may be configured to be coupled to the second end 132 of the handle 130 via a luer lock mechanism. A syringe tip 126 may be inserted into the first lumen 133 of the second portion 136 of the handle 130, and the syringe tip 126 may abut a first valve 137a. The first valve 137a may be positioned within the first lumen 133. As shown in FIG. 10B, when the syringe 120 is coupled to the second end 132 of the handle 130, the syringe tip 126 is inserted into the first lumen 133, which may force the first valve 137a open to allow a fluid or gel to pass therethrough into the first lumen 133, then subsequently the second lumen 138, the third lumen 139, and the lumen 145 of the delivery needle 40. The first valve 137a may be configured to be a self-sealing valve and may be configured to provide a seal between the second end 132 of the handle 130 and the first lumen 133 when the syringe 120 is removed from the second end 132, thereby preventing air from entering the first lumen 133 and/or a fluid from exiting the first lumen 133.

FIG. 10C illustrates an enlarged view of the distal end 141 of the delivery needle 140 as in FIG. 10A, taken at circle 10C. As shown in FIG. 10C, the distal end 141 of the delivery needle 140 may be beveled to enhance tissue penetration. The delivery needle 140 may include the lumen 145, and the stylet 163 including the sensor cable 160 may extend distally through the lumen 145 of the delivery needle 140 such that the sensor 165 may be positioned near the distal end 141 of the delivery needle 140. In some cases, the delivery needle 140 may include an outer diameter D₁ in a range of about 0.035 inches to 0.150 inches. In some cases, the delivery needle 140 may include an outer diameter D₁ in a range of about 0.030 inches to 0.042 inches. In some cases, the delivery needle 140 may include an outer diameter D₁ of 0.050 inches. In some cases, the stylet 163 may include an outer diameter D₂ in a range of about 0.007 inches to 0.149 inches. In some cases, the stylet 163 may include an outer diameter D₂ of 0.036 inches. In some cases, the sensor 165 may include an outer diameter in a range of about 0.005 inches to 0.145 inches. In some cases, the sensor 165 may include an outer diameter D₃ in a range of about 0.012 inches to 0.022 inches. In some cases, the sensor 165 may include an outer diameter D₃ of 0.018 inches.

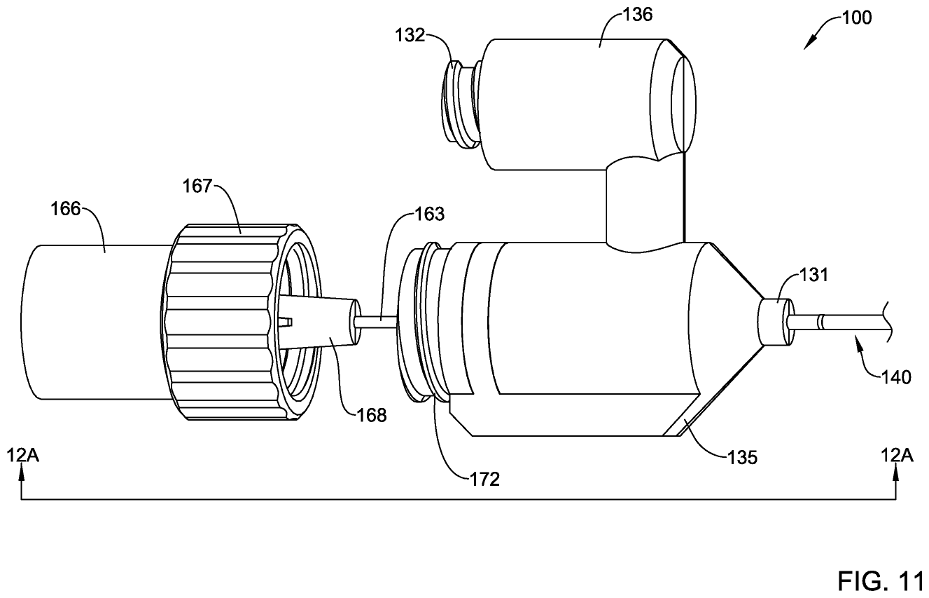

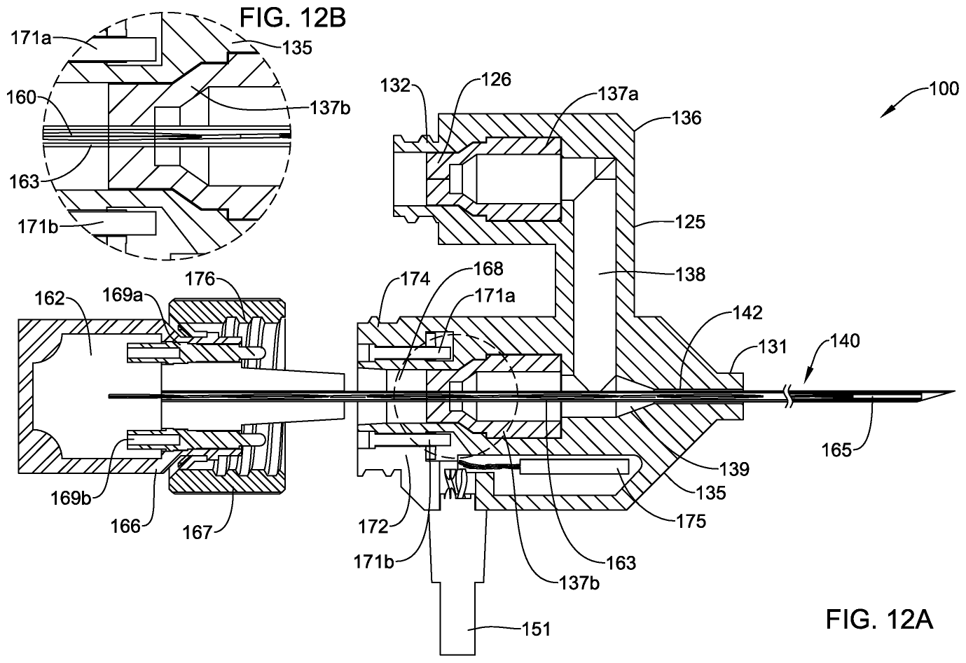

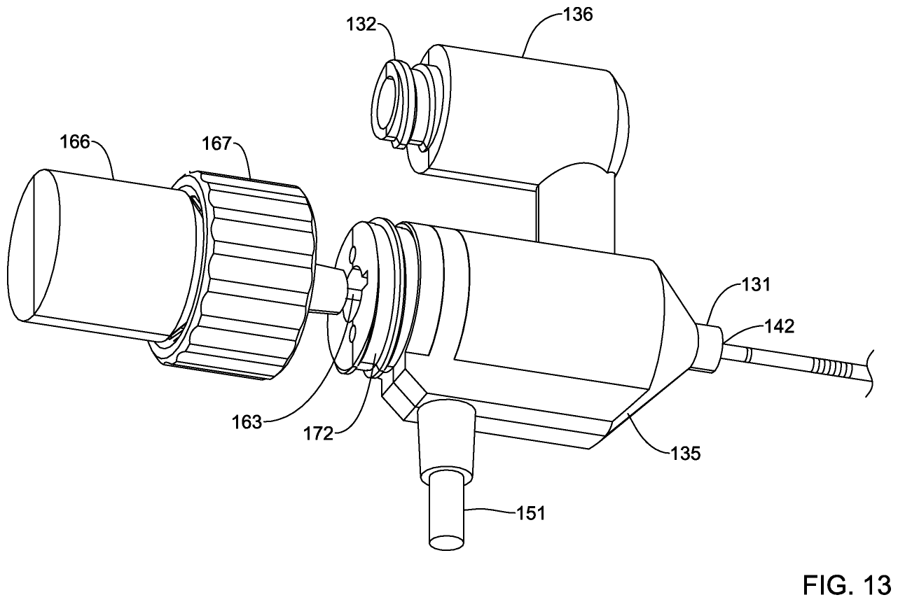

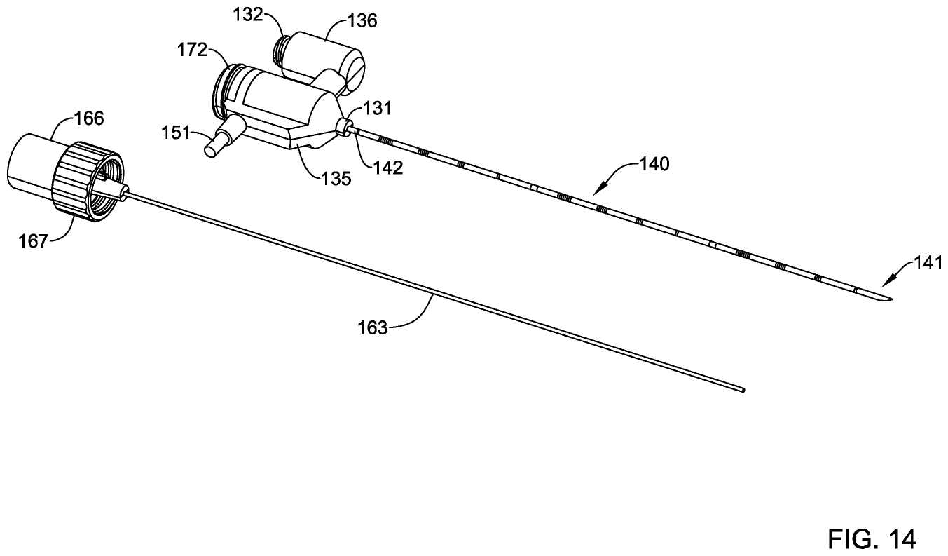

FIGS. 11 to 14 illustrate the removal of the adapter 166 from the handle 130, wherein the stylet 163 including the sensor cable 160 and the sensor 165 are retracted proximally from the delivery needle 140 and the handle 130 via the removal of the adapter 166. FIG. 11 illustrates a side view of the exemplary medical device 100 including the delivery needle 140, as in FIG. 7, wherein the adapter 166 has been disengaged from the handle 130, and FIG. 12A illustrates a cross-section view of the exemplary medical device 100 including the delivery needle 140, as in FIG. 11, taken at line 12A-12A. FIG. 13 illustrates a rear perspective view of the exemplary medical device 100 including the delivery needle 140, as in FIG. 7, wherein the adapter 166 is disengaged from the handle 130. FIG. 14 illustrates a front perspective view of the exemplary medical device 100 including the delivery needle 140, as in FIG. 14, wherein the adapter 166 is fully removed from the handle 130. As previously discussed, with reference to FIGS. 7 to 10A, the adapter 166 may be removably coupled to the outlet 172 of the first portion 135 of the handle 130 via a thread fastening mechanism. The outlet 172 may include the outer threads 174 which may be configured to engage with the inner threads 176 on the outer ring 167 of the adapter 166. The inner threads 176 and the outer threads 174 may engage via thread fastening to couple the adapter 166 to the first portion 135 of the handle 130. In some cases, the outlet 172 and the outer ring 167 may not include threads and may instead couple the adapter 166 and the first portion 135 of the handle 130 via interference fit, a snap-fit, adhesive, or any other suitable method of coupling. In some cases, the adapter 166 and the handle 130 may be formed as a single monolithic piece.

When the user uncouples the adapter 166 from the first portion 135 of the handle 130, the user may twist the outer ring 167 of the adapter 166 in direction such that the inner threads 176 and the outer threads 174 may uncouple. A user may pull the adapter 166 in a proximal direction, which may then retract the stylet 163 including the sensor cable 160 and the sensor 165 proximally from the delivery needle 140 and the handle 130. A user may continue to pull the adapter 166 in the proximal direction until the stylet 163 including the sensor cable 160 and the sensor 165 are fully removed (e.g., retracted) from the handle 130. Once the stylet 163 including the sensor cable 160 and the sensor 165 are fully removed, a user may inject a fluid (e.g., saline or hydrogel) via the syringe 120 through the delivery needle 140 to a tissue of a patient. In some cases, when the adapter 166 is removed from the outlet 172, a second fluid delivery device may be coupled to the outlet 172 (although not shown). In some cases, when the adapter 166 is removed from the outlet 172, a cap may be coupled to the outlet 172 (although not shown).

FIG. 12B illustrates an enlarged view of a portion of the second valve 137b, as in FIG. 12A, taken at circle 12B. As discussed with reference to FIG. 10A, the tip 168 may be inserted into the third lumen 139 and may abut the second valve 137b that is positioned within the third lumen 139. The stylet 163 including the sensor cable 160 may be configured to pass through the second valve 137b that is positioned within the third lumen 139. The second valve 137b may be configured to provide a seal around the stylet 163 including the sensor cable 160, thereby preventing air from entering the third lumen 139 and/or a fluid from exiting the third lumen 139. In some cases, the second valve 137b may be a self-sealing valve, and may be configured to provide a seal between the outlet 172 and the third lumen 139 upon removal of the adapter 166, and thus the stylet 163 including the sensor cable 160 and the sensor 165.

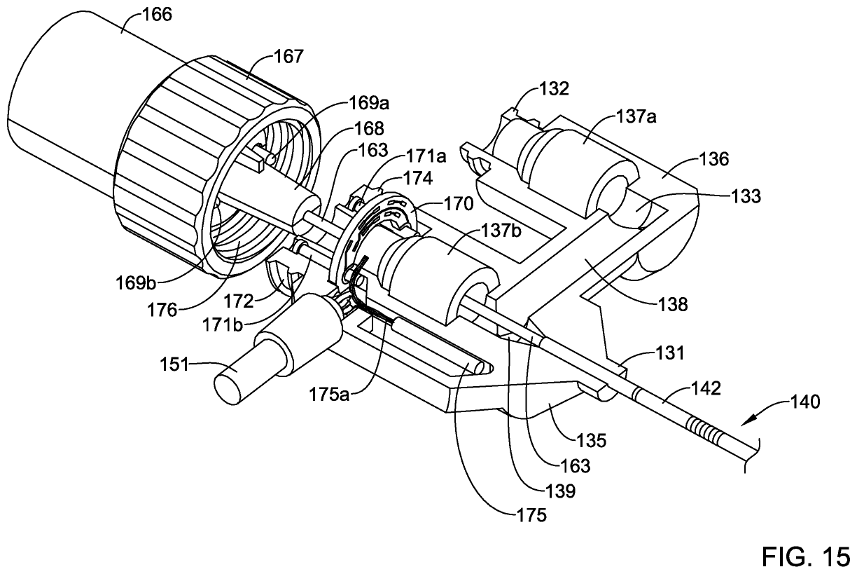

FIG. 15 illustrates a partial cross-section view of the exemplary medical device 100 including the delivery needle 140, as in FIG. 11, wherein the handle 130 is shown in cross-section. As shown in FIG. 15, an interposer board 170 may be positioned within the first portion 135 of the handle 130. The interposer board 170 may be configured to be coupled to the second sensor 175 and the electrical connector 151 via one or more wires 175a. The one or more wires 175a may be soldered to the interposer board 170 to enhance assembly of the handle 130. The adapter 166 may include a grounding tab 179 which may serve to control polarity within the handle 130. When the handle 130 is fully assembled, the interposer board 170 may be sealed within the handle 130 out of a fluid path, e.g., the third lumen 139.

The medical device 10, 100, or parts thereof, may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all of medical device 10, 100, or parts thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of medical device 10, 100, or parts thereof, in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of medical device 10, 100, or parts thereof, to achieve the same result.

In some embodiments, a degree of Magnetic Resonance Imaging (MRI) compatibility is imparted into medical device 10, 100, or parts thereof. For example, medical device 10, 100, or parts thereof, may be made of a material that does not substantially distort the image and create substantial artifacts (i.e., gaps in the image). Certain ferromagnetic materials, for example, may not be suitable because they may create artifacts in an MRI image. The medical device 10, 100, or parts thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

This disclosure is, in many respects, only illustrative. Changes may be made in detail, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical device comprising:
a handle;
an actuation mechanism coupled to the handle;
a delivery needle having a distal end, a proximal end, and a lumen extending from the distal end to the proximal end, wherein the proximal end of the delivery needle is coupled to a first end of the handle;
a fluid delivery device coupled to a second end of the handle; and
a retractable sensor cable positioned within the handle, the retractable sensor cable configured to extend distally through the lumen of the delivery needle in a non-retracted position during placement of the delivery needle within a body of a patient;
wherein actuation of the actuation mechanism causes the retractable sensor cable to retract proximally through the lumen of the delivery needle into the handle to a retracted position.

2. The medical device of claim 1, wherein when the retractable sensor cable is in the retracted position, the retractable sensor cable is wound on a spool contained within the handle.

3. The medical device of claim 1, wherein the fluid delivery device is a syringe.

4. The medical device of claim 1, wherein a sensor is positioned at a distal end of the retractable sensor cable.

5. The medical device of claim 2, wherein when the retractable sensor cable is in the non-retracted position, one or more tabs engage with the spool to lock the spool and thereby the retractable sensor cable in the non-retracted position.

6. The medical device of claim 5, wherein actuation of the actuation mechanism unlocks the spool, causing the retractable sensor cable to retract proximally and wind around the spool within the handle to the retracted position.

7. The medical device of claim 1, wherein the retractable sensor cable is a flex-circuit cable.

8. The medical device of claim 4, wherein the sensor has an outer diameter in a range of Ø.005 inches to Ø.145 inches.

9. The medical device of claim 1, wherein upon retraction of the retractable sensor cable, the fluid delivery device is configured to deliver a fluid through the lumen of the delivery needle.

10. The medical device of claim 4, wherein the sensor is a position sensor.

11. The medical device of claim 1, wherein the actuation mechanism is a button.

12. A medical device comprising:
a handle;
an actuation mechanism coupled to the handle;
a delivery needle having a distal end, a proximal end, and a lumen extending from the distal end to the proximal end, wherein the proximal end of the delivery needle is coupled to a first end of the handle;
a retractable sensor cable positioned within the handle, the retractable sensor cable configured to extend distally through the lumen of the delivery needle in a non-retracted position during placement of the delivery needle within a body of a patient; and
a spool positioned within the handle, the retractable sensor cable coupled to the spool;
wherein actuation of the actuation mechanism causes the retractable sensor cable to retract proximally through the lumen of the delivery needle into the handle and wind around the spool in a retracted position.

13. The medical device of claim 12, wherein when the retractable sensor cable is in the non-retracted position, one or more tabs engage with the spool to lock the spool and thereby the retractable sensor cable in the non-retracted position.

14. The medical device of claim 13, wherein actuation of the actuation mechanism unlocks the spool, causing the retractable sensor cable to retract proximally and wind around the spool within the handle to the retracted position.

15. The medical device of claim 12, wherein the actuation mechanism is a button.

16. The medical device of claim 12, wherein a sensor is positioned at a distal end of the retractable sensor cable.

17. A medical device comprising:
a handle;
an actuation mechanism coupled to the handle;
a delivery needle having a distal end, a proximal end, and a lumen extending from the distal end to the proximal end, wherein the proximal end of the delivery needle is coupled to a first end of the handle; and
a retractable sensor cable positioned within the handle, the retractable sensor cable configured to extend distally through the lumen of the delivery needle in a non-retracted position during placement of the delivery needle within a body of a patient.

18. The medical device of claim 17, wherein actuation of the actuation mechanism causes the retractable sensor cable to retract proximally through the lumen of the delivery needle into the handle to a retracted position.

19. The medical device of claim 17, wherein the actuation mechanism is a button.

20. The medical device of claim 18, wherein when the retractable sensor cable is in the retracted position, the retractable sensor cable is wound on a spool contained within the handle.
